(19) United States
(12) Patent Application Publication
(10) Pub. No.: US 2019/0150921 A1
(43) Pub. Date: May 23, 2019

(54) STAPLE DELIVERY DEVICE

(71) Applicant: Arthrex, Inc., Naples, FL (US)

(72) Inventors: Matthew Fonte, Concord, MA (US); Robert Devaney, Auburndale, MA (US); Alexander DelMonaco, Billerica, MA (US); Paul Fein, Melrose, MA (US)

(21) Appl. No.: 15/820,826

(22) Filed: Nov. 22, 2017

(51) Int. Cl. A61B 17/068; A61B 17/064

(52) U.S. Cl. CPC A61B 17/0684; A61B 2017/564; A61B 2017/0645; A61B 17/0642

(57) ABSTRACT

Staple delivery devices are disclosed herein that include a plunger that translate to engage an elastic bridge of a staple to pivot legs of the staple outwardly to a strained state. Two or more pins retain the staple to the delivery device.

### BACKGROUND

This disclosure relates to staple delivery devices.

### SUMMARY

A delivery device strains a staple for insertion of the staple to set two or more bone fragments (e.g., bone fracture, osteotomy, etc.). The staple includes an elastic bridge and at least two legs each connected to the elastic bridge at a hinge region. The staple is loaded into/onto the delivery device in a non-strained state. When the delivery device is activated, a plunger applies pressure to the elastic bridge to pivot the legs outwardly to a strained state for insertion at the bone fracture site.

Embodiments of staple delivery devices disclosed herein include a plunger that translates distally to engage an elastic bridge of a staple to pivot legs of the staple outwardly to a strained state. Legs, 2 or more, of a staple are each connected to a bridge at a hinge region. The delivery device includes at least two pivotable arms, each including a pin. The pins can be received under the bridge or the hinge regions of a staple or in a hole in the hinge region of the staple to relesably mount the staple to the delivery device. A plunger can also be translated by actuation of a lever including a cam that engages the plunger.

In an embodiment, a delivery device includes a body, a lever including a cam at a distal end of the lever, and a plunger. When the plunger is advanced into the body by actuation of the lever, the cam pushes against a proximal end of the plunger to translate the plunger, and a distal end of the plunger is configured to engage and bend an elastic bridge of a staple to a strained state.

In another embodiment, an assembly includes a staple including an elastic bridge and at least two elastic legs, wherein the legs meet the bridge at a curved, elastic hinge region. The assembly includes a delivery device including a housing supporting two moveable pins. The pins are configured to relesably receive a staple. A handle is actuated in a longitudinal direction to selectively translate a plunger towards the pins. The staple is mounted on the pins.

In another embodiment, a method of installing a staple to maintain compression between bone fragments includes pivoting a lever of a delivery device to engage a cam of the lever against a plunger. The method also includes translating the plunger distally to engage and bend an elastic bridge of the staple to a strained state.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts, and further wherein:

FIG. 1 is a schematic view of an embodiment of a staple in a non-strained state;

FIG. 2 is a schematic view of an embodiment of the staple in a strained state;

FIG. 3 is a schematic view of an embodiment of the staple moving between the strained state and the non-strained state;

FIG. 4 is a front view of an embodiment of a first delivery device with the staple in the non-strained state;

FIG. 5A is a front view of an embodiment of the first delivery device with the staple in the strained state;

FIG. 5B is a rear view of an embodiment of the first delivery device with the staple in the strained state;

FIG. 6A is a view of an embodiment of a second delivery device with the staple in the non-strained state;

FIG. 6B is a view of an embodiment of the second delivery device with the staple in the strained state and a portion of a body removed;

FIG. 7A is a view of an embodiment of the second delivery device with the staple in the non-strained state;

FIG. 7B is a view of an embodiment of the second delivery device with the staple in the strained state and a portion of the body removed;

FIG. 8 is a front view of an embodiment of a third delivery device with the staple in the non-strained state and pins located outside the legs of the staple; and

FIG. 9 is a schematic view of an embodiment showing installation of the staple to generate and maintain compression between bone fragments.

### DETAILED DESCRIPTION

A delivery device strains a staple for insertion of the staple to set two or more bone fragments (e.g., bone fracture, osteotomy, etc.). The staple includes an elastic bridge and at least two legs each connected to the elastic bridge at a hinge region. A staple is loaded onto a delivery device in a non-strained state. When a delivery device is activated, a plunger applies pressure to an elastic bridge to pivot staple legs outwardly to a strained state for insertion between bone fragments.

Staple delivery devices are disclosed herein that include a plunger that translates distally to engage an elastic bridge of a staple to pivot legs of the staple outwardly to a strained state. The legs of the staple are each connected to the bridge at a hinge region. The delivery device includes two pivotable arms each including a pin. The pins can be received a) under i) the bridge or ii) the hinge regions of a staple or b) in a hole in the hinge region of the staple to releasable mount the staple to the delivery device. The plunger can also be translated by actuation of a lever including a cam that engages the plunger. Pressing a handle of a delivery device to translate the plunger allows for faster installation of the staple in the bone.

In an embodiment, a delivery device includes a body, a lever including a cam at a distal end of the lever, and a plunger. When the plunger is advanced into the body by actuation of the lever, the cam pushes against a proximal end of the plunger to translate the plunger, and a distal end of the plunger is configured to engage and bend an elastic bridge of a staple to a strained state.

In an embodiment, the lever is pivoted about a fulcrum to engage the cam with the proximal end of the plunger to advance the plunger distally. In an embodiment, two arms are pivotably attached to the body, and each arm includes a pin.

In another embodiment, an assembly includes a staple including an elastic bridge and at least two elastic legs, and the legs meet the bridge at a curved, elastic hinge region. The assembly includes a delivery device including a housing supporting two moveable pins. The pins are configured to relesably receive a staple. A handle is actuated in a longitudinal direction to selectively translate a plunger towards the pins. The staple is mounted on the pins.

In an embodiment, the handle actuates the plunger in the distal direction to engage the elastic bridge of the staple. In an embodiment, two arms are pivotably attached to the body, and each arm includes one of the two pins. In an embodiment, the staple consists of Nitinol. In an embodiment, the staple comprises Nitinol. In an embodiment, the staple consists essentially of Nitinol. In an embodiment, the staple comprises polyether ether ketone (PEEK). In an embodiment, strain on the bridge of the staple bends the bridge into a more linear configuration and bends the two legs to be parallel.

In another embodiment, a method of installing a staple to maintain compression between bone fragments includes pivoting a lever of a delivery device to engage a cam of the lever against a plunger. The method can also include translating the plunger distally to engage and bend an elastic bridge of the staple to a strained state.

In an embodiment, the delivery device includes two arms pivotably attached to the body, and each of the two arms includes a pin. The staple includes two legs that each attach to the elastic bridge of the staple at a hinge region.

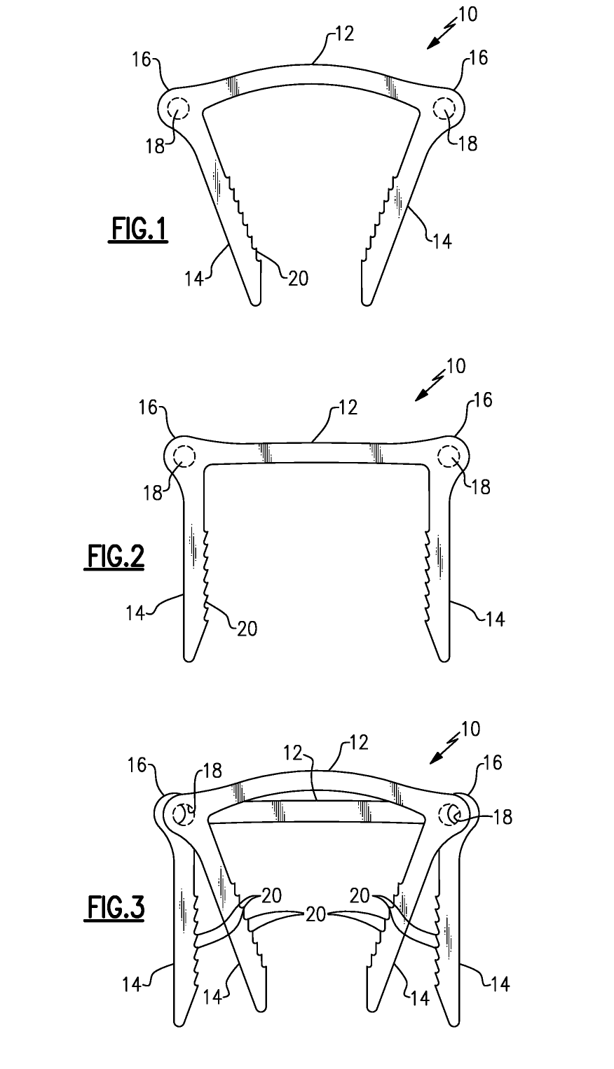

As shown in FIGS. 1 and 2, a staple 10 includes an elastic bridge 12 and two elastic legs 14. The bridge 12 meets each of the legs 14 at one of two curved hinge regions 16, which are also elastic. In one exemplary embodiment, an inner surface of the legs 14 include barbed teeth 20 to help the legs 14 grip a bone after implantation and to prevent the legs 14 from working their way out of the bone. In one exemplary embodiment, the staple 10 includes more than two legs 14.

In an exemplary embodiment, the hinge regions 16 each include a hole 18 (shown in dotted lines in FIGS. 1 to 3). In one exemplary embodiment, the holes 18 are round. However, the holes 18 can have any shape. In another exemplary embodiment, the hinge regions 16 do not include any holes.

In one exemplary embodiment, the staple 10 is manufactured from a shape memory material (for example, a material capable of exhibiting superelasticity and/or a temperature-induced shape change). The shape memory material may include a metal alloy or a polymer. In one exemplary embodiment, the shape memory alloy is Nitinol. In one exemplary embodiment, the polymer is appropriately processed polyether ether ketone (PEEK). In one exemplary embodiment, the staple 10 may be manufactured from another suitable material, such as stainless steel, titanium, etc. The staple 10 can reduce bone fracture(s) and generate and maintain compression between bone fragments across a fracture line to aid in fracture healing.

As shown in FIG. 1, in a non-strained state, the bridge 12 is bowed upwardly. That is, the bridge 12 has a convex configuration. In the non-strained state, the legs 14 of the staple 10 are elastically pivoted inwardly at the elastic hinge regions 16 at an angle of less than 90° relative to a longitudinal axis the bridge 12. In one exemplary embodiment, the legs 14 extend at an angle of about 65° to the longitudinal axis of the bridge 12 when in the non-strained state.

As shown in FIG. 2, prior to implantation, the bridge 12 of the staple 10 can be reversibly bent (that is, the bridge 12 can be bent to be nearly linear), and the legs 14 of the staple 10 can be reversibly pivoted at the elastic hinge regions 16 to be substantially perpendicular to the bridge 12. That is, in the retrained state, the legs 14 are parallel. This allows for insertion of the legs 14 in bone fragments, and the bridge 12 spans a space between the bone fragments. If the staple 10 is formed out of Nitinol, elastic deformations of up to approximately 8% are achievable.

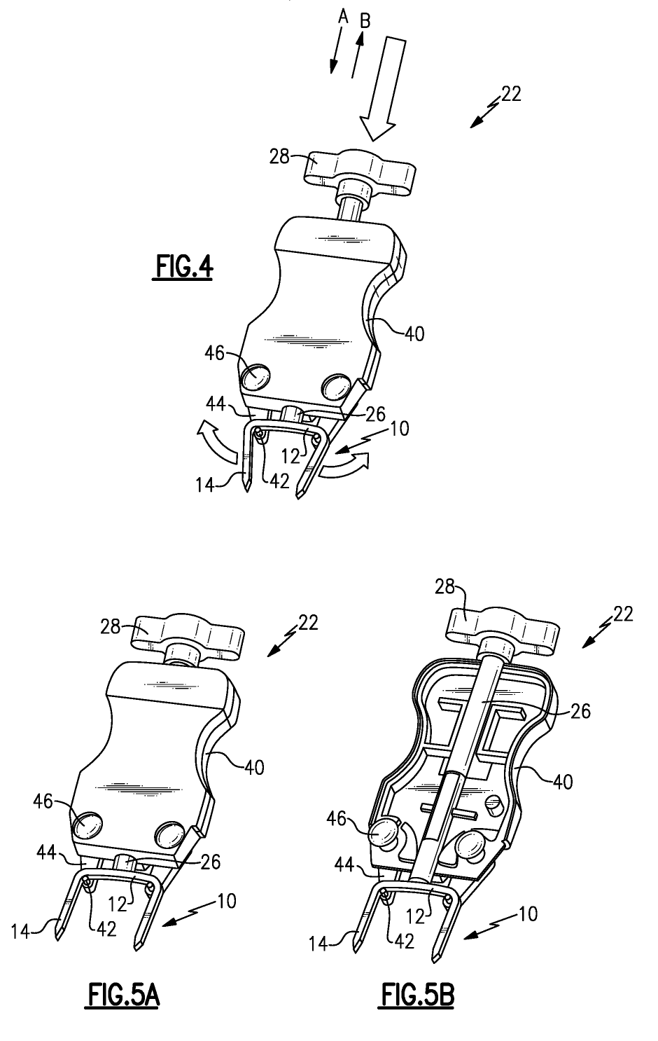

As shown in FIG. 3, upon insertion of the strained staple 10 in bone fragments, the constraint on the bridge 12 is removed, and the legs 14 move outwardly as the staple 10 attempts to return to the original non-strained state. This generates and maintains a compressive load across a space between the bone fragments.

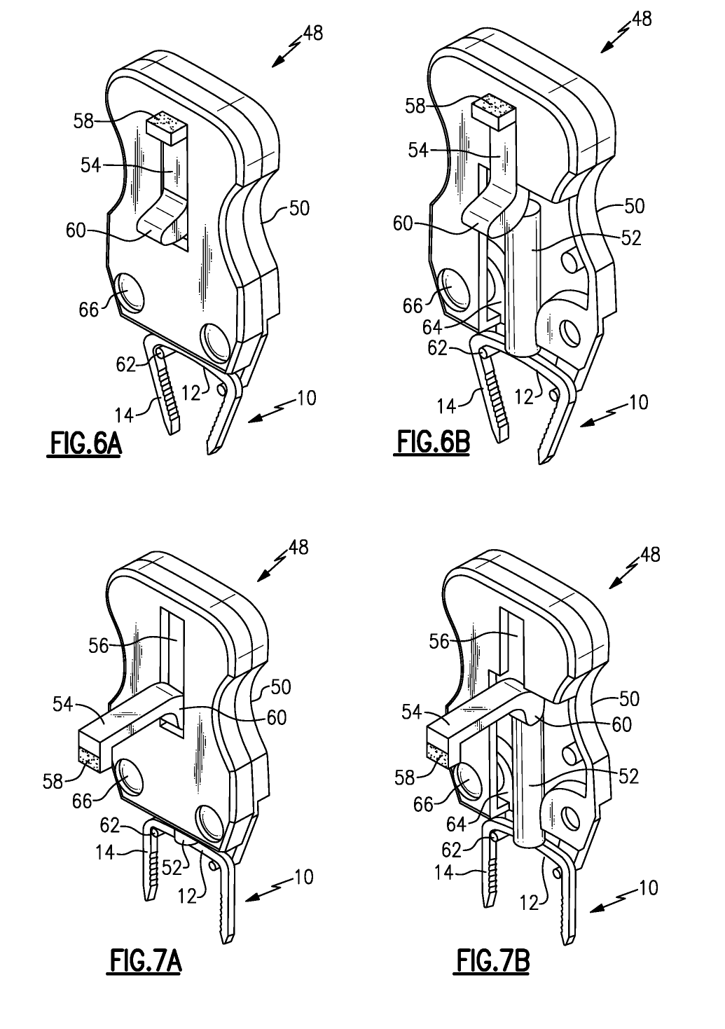

FIGS. 4, 5A, and 5B show an exemplary embodiment of a delivery device 22 that can be used to selectively bend the bridge 12 of the staple 10 for installation of the staple 10 to set bone fragments. The delivery device 22 elastically bends the bridge 12 to pivot the legs 14 outwardly at the elastic hinge regions 16, constrains and holds the staple 10 in the strained state prior to implantation, and then inserts the staple 10 into a fracture site.

The delivery device 22 includes a body 24 and a plunger 26. A handle 28 is attached to a proximal end of the plunger 26. Initially, the staple 10 is in the non-strained state as shown in FIG. 4. When the plunger 26 is advanced into the body 24 by selectively pushing the handle 28 distally in a direction A, a distal end of the plunger 26 pushes against the bridge 12 of the staple 10 and elastically bends the bridge 12 to a more linear configuration such that the staple 10 is in the strained state and the legs 14 are parallel (shown in FIGS. 5A and 5B).

In one exemplary embodiment, the staple 10 is releasably mounted to the delivery device 22 by two pins 42. Each pin 42 is attached to an arm 44 of the delivery device 22, and each arm 44 is pivotally mounted to the body 24 of the delivery device 22 by a pivot pin 46. The pins 42 can be received under the bridge 12 or the hinge regions 16 of a staple 10.

When the plunger 52 is moved distally and presses against the elastic bridge 12 of the staple 10, the bridge 12 deforms, causing the arms 44 of the delivery device 22 to pivot outwardly. The elastic hinge regions 16 of the staple 10 move outwardly and bend about the pins 42 to pivot the legs 14 outwardly about the elastic hinge regions 16 so that the legs 14 are oriented generally perpendicular to the elastic bridge 12. As the elastic hinge regions 16 move outwardly, the pins 42 also move outwardly, causing the attached arms 44 to pivot outwardly about the pivot pins 46.

The staple 10 can be released from the delivery device 22 by removing the pins 42 from under the bridge 12 or the hinge regions 16 and pulling the handle 28 in an opposing direction B, moving the plunger 26 proximally and away from the staple 10.

The pressure that the surgeon applies to the handle 28 can control the position of the legs 14 of the staple 10. That is, spacing between the legs 14 of the staple 10 is proportional to the pressure applied to the handle 28. Additionally, the amount of pressure needed to actuate the handle 28 also provides tactile feedback to the surgeon.

The delivery device 22 can also be used to remove the staple 10 from bone. The pins 42 of the delivery device 22 are inserted under the bridge 10 or the hinge regions 16 of the staple 10. The handle 28 can then be pulled in the opposite direction B to move the plunger 52 proximally and to reconfigure the staple 10 such that the bridge 12 is substantially perpendicular to the legs 14. The delivery device 22 can be pulled away from the bone to remove the staple 10 from the bone.

In another exemplary embodiment, the pins 42 are received within the holes 18 in the staple 10. As the plunger 52 engages the bridge 12 of the staple, the elastic hinge regions 16 and the attached pins 42 also move outwardly, causing the attached arms 44 to pivot outwardly about the pivot pins 46. The staple 10 can be released from the delivery device 22 by removing the pins 42 from the holes 18 in the staple 10 and pulling the handle 28 in an opposing direction B, moving the plunger 26 proximally and away from the staple 10. The delivery device 22 can also be used to remove the staple 10 from bone. The pins 42 of the delivery device 22 are inserted in the holes 18 of the hinge regions 16 of the staple 10. The handle 28 can then be pulled in the opposite direction B to move the plunger 52 proximally and to reconfigure the staple 10 such that the bridge 12 is substantially perpendicular to the legs 14. The delivery device 22 can be pulled away from the bone to remove the staple 10 from the bone.

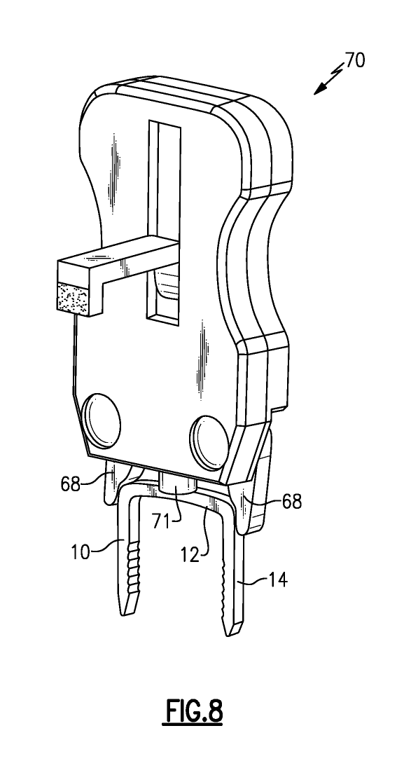

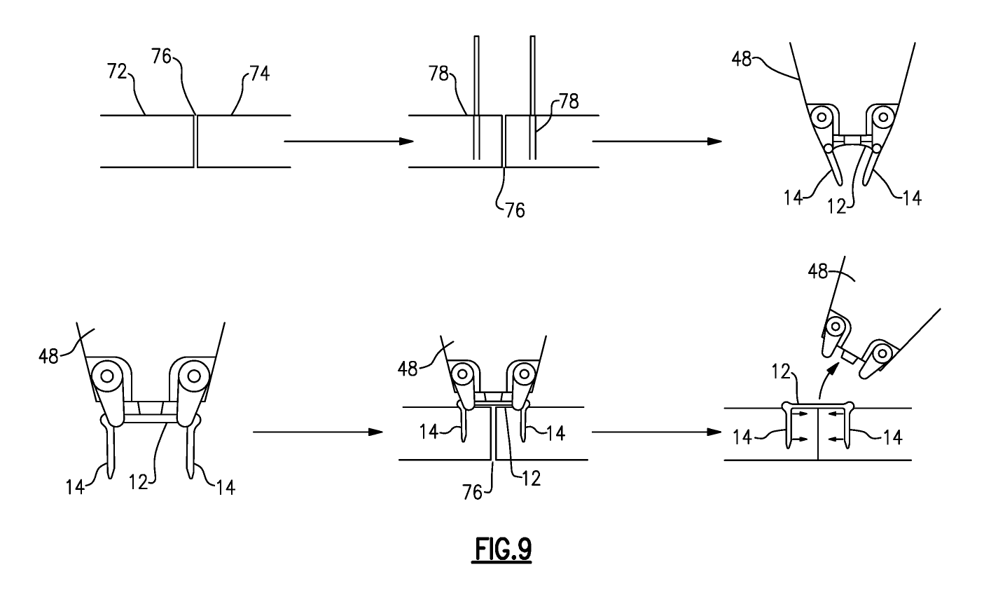

FIGS. 6A, 6B, 7A, and 7B show an exemplary embodiment of a delivery device 48 that can be used to selectively bend the bridge 12 of the staple 10 and to selectively pivot the legs 14 of the staple 10 at the elastic hinge regions 16 of the staple 10. The delivery device 48 elastically bends the bridge 12 to pivot the legs 14 outwardly at the elastic hinge regions 16, constrains and holds the staple 10 in the strained state prior to implantation, and then inserts the staple 10 to set bone fragments.

The delivery device 48 includes a body 50, a plunger 52, and a lever 54. The lever 54 includes a gripping portion 58 at one end and a cam 60 at an opposing end that interfaces with the plunger 52. Initially, the staple 10 is in a non-strained state as shown in FIGS. 6A and 6B. When the lever 54 is pivoted about a fulcrum by selectively moving the lever 54 by moving the gripping portion 58, the mechanical advantage of the lever 54 applies force to the plunger 52 through the cam 60 to advance the plunger 52 distally in the body 50. The plunger 52 pushes against the bridge 12 of the staple 10 and elastically bends the bridge 12 into a more linear configuration such that the staple 10 is in a strained state and the legs 14 are parallel (shown in FIGS. 7A and 7B).

In one exemplary embodiment, the staple 10 is releasably mounted to the delivery device 48 by two pins 62. Each pin 62 is attached to an arm 64 of the delivery device 48, and each arm 64 is pivotally mounted to the body 50 of the delivery device 48 by a pivot pin 66. The pins 62 are located under the bridge 12 or the hinge regions 16 of the staple 10.

The lever 54 is pivoted to engage the cam 60 with the plunger 52 and move the plunger 52 distally towards the staple 10. The plunger 52 presses against the elastic bridge 12 of the staple 10, deforming the bridge 12 to cause the arms 64 of the delivery device 48 to pivot outwardly. The elastic hinge regions 16 of the staple 10 move outwardly and bend about the pins 62 to pivot the legs 14 outwardly about the elastic hinge regions 16 so that the legs 14 are oriented generally perpendicular to the elastic bridge 12. As the elastic hinge regions 16 move outwardly, the pins 62 also move outwardly, causing the arms 64 to pivot outwardly about the pivot pins 66.

The staple 10 can be released from the delivery device 48 by removing the pins 62 from under the bridge 12 or the hinge regions 16 of the staple 10 and pivoting the lever 54 back to an original position, moving the plunger 52 proximally away from the staple 10.

The pressure that the surgeon applies to the lever 54 can also control the position of the legs 14 of the staple 10. That is, spacing between the legs 14 of the staple 10 is proportional to the pressure applied to the lever 54 by the surgeon. Additionally, the amount of pressure needed to actuate the lever 54 also provides tactile feedback to the surgeon.

The delivery device 48 can also be used to remove the staple 10 from the bone. The pins 62 of the delivery device 48 are located under the bridge 12 or the hinge regions 16 of the staple 10. The lever 54 can then be pivoted back to the original position to cause the plunger 52 to move proximally, reconfiguring the staple 10 such that the bridge 12 is substantially perpendicular to the legs 14. The delivery device 48 can be pulled away from the bone to remove the staple 10 from the bone.

In one exemplary embodiment, the pins 62 are received within the holes 18 in the staple 10. The plunger 52 presses against the elastic bridge 12 of the staple 10, deforming the bridge 12 to cause the arms 64 of the delivery device 48 to pivot outwardly. The elastic hinge regions 16 of the staple 10 move outwardly and bend about the pins 62 to pivot the legs 14 outwardly about the elastic hinge regions 16 so that the legs 14 are oriented generally perpendicular to the elastic bridge 12. As the elastic hinge regions 16 move outwardly, the attached pins 62 also move outwardly, causing the attached arms 64 to pivot outwardly about the pivot pins 66. The staple 10 can be released from the delivery device 48 by removing the pins 62 from the holes 18 in the staple 10 and pivoting the lever 54 back to an original position, moving the plunger 52 proximally away from the staple 10. The delivery device 48 can also be used to remove the staple 10 from the bone. The pins 62 of the delivery device 48 are inserted in the holes 18 of the hinge regions 16 of the staple 10. The lever 54 can then be pivoted back to the original position to move the plunger 52 proximally, reconfiguring the staple 10 such that the bridge 12 is substantially perpendicular to the legs 14. The delivery device 48 can be pulled away from the bone to remove the staple 10 from the bone.

FIG. 8 shows another example delivery device 70 including arms 68 that are biased inwardly. The staple 10 is located between the two arms 68 and releasably mounted to the delivery device 70 by inward pressure applied by the arms 68. The arms 68 press on the legs 14 of the staple 10 to retain the staple 10 between the arms 68 and to the delivery device 48. When a plunger 71 advances distally to engage the bridge 12, the staple 10 is released from the delivery device 48.

In one exemplary embodiment, the staple 10 is retained to the delivery device by an arm that grips under the bridge 12 of the staple 10 (not shown).

FIG. 9 shows the staple 10 installed in a bone to assist in healing a fracture 76 and to generate and maintain compression between bone fragments 72 and 74. The fracture 76 can first be re-approximated and reduced. A drill guide (not shown) is used to drill two holes 78 a distance apart to accommodate the legs 14 of the staple 10. The staple 10 is mounted to the pins 42 of the delivery device 48, and the lever 54 of the delivery device 48 is pivoted to translate the plunger 52 to engage and bend the bridge 12 of the staple 10 and straighten the legs 14 of staple 10. The legs 14 of the staple 10 are placed into the pre-drilled holes 78. The staple 10 is then released from the pins 42 of the delivery device 48 by pivoting the lever 54 in the opposite direction B and removing the staple 10 from the pins 42. When in the bone, the bridge 12 and the legs 14 of the staple 10 attempt to return to the original configuration, applying compression across the fracture 76. During healing, the bridge 12 is in direct contact with the bone fragments 72 and 74.

The foregoing description is only exemplary of the principles of the invention. Many modifications and variations are possible in light of the above teachings. It is, therefore, to be understood that within the scope of the appended claims, the invention may be practiced otherwise than using the example embodiments which have been specifically described. For that reason the following claims should be studied to determine the true scope and content of this invention.

What is claimed is:

1. A delivery device including:
a) a body;
b) a lever including a cam at a distal end of the lever; and
c) a plunger, wherein when the plunger is advanced into the body by actuation of the lever,
i) the cam pushes against a proximal end of the plunger to translate the plunger, and
ii) a distal end of the plunger is configured to engage and bend an elastic bridge of a staple to a strained state.

2. The delivery device of claim 1 wherein the lever is pivoted about a fulcrum to engage the cam with the proximal end of the plunger to advance the plunger distally.

3. The delivery device of claim 1 including two arms pivotably attached to the body, wherein each arm includes a pin.

4. An assembly including:
a) a staple including an elastic bridge and two elastic legs, wherein the legs meet the bridge at a curved, elastic hinge region; and
b) a delivery device including a housing supporting two moveable pins, wherein the pins are configured to releasably receive the staple, and a handle is actuated in a longitudinal direction to selectively translate a plunger towards the two pins,
wherein when the staple is mounted on the two pins.

5. The assembly of claim 4 wherein the handle actuates the plunger in a distal direction to engage the elastic bridge of the staple.

6. The assembly of claim 4 including two arms pivotably attached to the body, and each of the two arms includes one of the two pins.

7. The assembly of claim 4 wherein the staple consists of Nitinol.

8. The assembly of claim 4 wherein the staple comprises Nitinol.

9. The assembly of claim 4 wherein the staple comprises polyether ether ketone (PEEK).

10. The assembly of claim 4 wherein strain on the bridge of the staple bends the bridge into a more linear configuration and bends the two legs to be parallel.

11. A method of installing a staple to maintain compression between bone fragments comprising:
pivoting a lever of a delivery device to engage a cam of the delivery device against a plunger; and
translating the plunger distally to engage and bend an elastic bridge of the staple to a strained state.

12. The method as recited in claim 11 wherein the delivery device includes two arms pivotably attached to the body, and each of the two arms includes a pin to relesably mount the staple, and the staple includes two legs that each attach to the elastic bridge of the staple at a hinge region.
